(19) United States
(12) Patent Application Publication
(10) Pub. No.: US 2010/0234769 A1
(43) Pub. Date: Sep. 16, 2010

(54) Sports training system

(75) Inventors: Marius Octavian Poliac, Reno, NV (US); Roger C. Thede, Afton, MN (US); Donald A. Chu, Alameda, CA (US); Ross Andrew Rosemark, Eagan, MN (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: GFXCoach LLC, Reno, NV (US)

(21) Appl. No.: 12/660,972

(22) Filed: Mar. 8, 2010

Related U.S. Application Data
(60) Provisional application No. 61/209,875, filed on Mar. 11, 2009.

(51) Int. Cl. A61B 5/103; G06K 9/00

(52) U.S. Cl. 600/592; 382/100

(57) ABSTRACT

A method for optimizing the performance of an athlete includes capturing an image of the athlete performing a biomechanical movement. A video signal is generated from the captured image. Ground reaction forces generated by the athlete are measured at the athlete's insoles and are transmitted as force data to a central processing unit. The force data is synchronized with the video signal by the central processing unit for display or storage.

### CROSS-REFERENCE TO RELATED APPLICATION(S)

The present application claims priority to U.S. Provisional Pat. App. Ser. No. 61/209,875 entitled SPORTS TRAINING SYSTEM filed Mar. 11, 2009, which is hereby incorporated by reference in its entirety

### BACKGROUND

The present invention relates to a sports training system, and more particularly, to a method and assembly for capturing ground reaction forces produced by an athlete during training.

Athletic trainers, coaches, and sports medicine professionals have recognized that an athlete's biomechanics are critical to athletic health and performance. For example, in baseball, the athlete's overhand throwing motion requires contributions from the lower extremities as well as the throwing arm. Studies have linked arm mechanics of pitchers with the magnitude of shear forces generated by the push-off leg and the resistance force provided by the landing leg. These ground reaction forces are directly related to the ultimate velocity that pitchers develop while throwing. Thus, by strengthening their lower extremities to generate and withstand greater ground reaction forces, pitchers can enhance their performance and avoid injuries.

To date, force plates have been used to measure ground reaction forces generated by the athlete. Unfortunately, force plates are bulky, obtrusive, and relatively immobile. Force plates also do not adequately simulate the playing environment of the athlete. Additionally, little effort has been made to measure the ground reaction forces generated by the athlete while simultaneously capturing the biomechanics of the athlete with a video recorder for the purpose of teaching the athlete how to enhance performance and minimize the risk of injury.

Therefore, there is a need for a mobile, unobtrusive, and environmentally adaptive device which can measure the ground reaction forces generated by the athlete. There is also a need for a system that can digitally display the biomechanics of the athlete and synchronize the measured ground reaction forces with the athlete's biomechanics.

### SUMMARY

In one aspect, an assembly for optimizing the performance of an athlete includes a video unit, a sensor assembly, a transmission device, and a main unit. The video unit is configured to capture an image of the athlete and generate a video signal thereof. The sensor assembly is worn in a shoe of the athlete to measure ground reaction forces generated by the athlete. The transmission device transmits force data representative of the measured ground reaction forces. The main unit is adapted to receive and synchronize the force data with the video signal.

In another aspect, a method for optimizing the performance of an athlete includes capturing an image of the athlete performing a biomechanical movement. A video signal is generated from the captured image. Ground reaction forces generated by the athlete are measured at the athlete's insoles and are transmitted as force data to a central processing unit. The force data is synchronized with the video signal by the central processing unit for display or storage.

### BRIEF DESCRIPTION OF THE DRAWINGS

FIG. 1 is a perspective view of one embodiment of an athletic training assembly that synchronizes video signal captured by a video unit with force data measured by a sensor assembly worn in the shoes of an athlete.

FIG. 1A is an enlarged view of one embodiment of a display of a main unit of the athletic training assembly shown in FIG. 1.

FIG. 2A is a block diagram illustrating the components of one embodiment of the athletic training assembly.

FIG. 2B is a block diagram illustrating the components of another embodiment of the athletic training assembly.

FIG. 2C is a block diagram showing the components of a third embodiment of the athletic training assembly.

FIG. 2D is a block diagram showing the components of a fourth embodiment of the athletic training assembly.

FIG. 2E is a block diagram showing the components of a fifth embodiment of the athletic training assembly.

### DETAILED DESCRIPTION

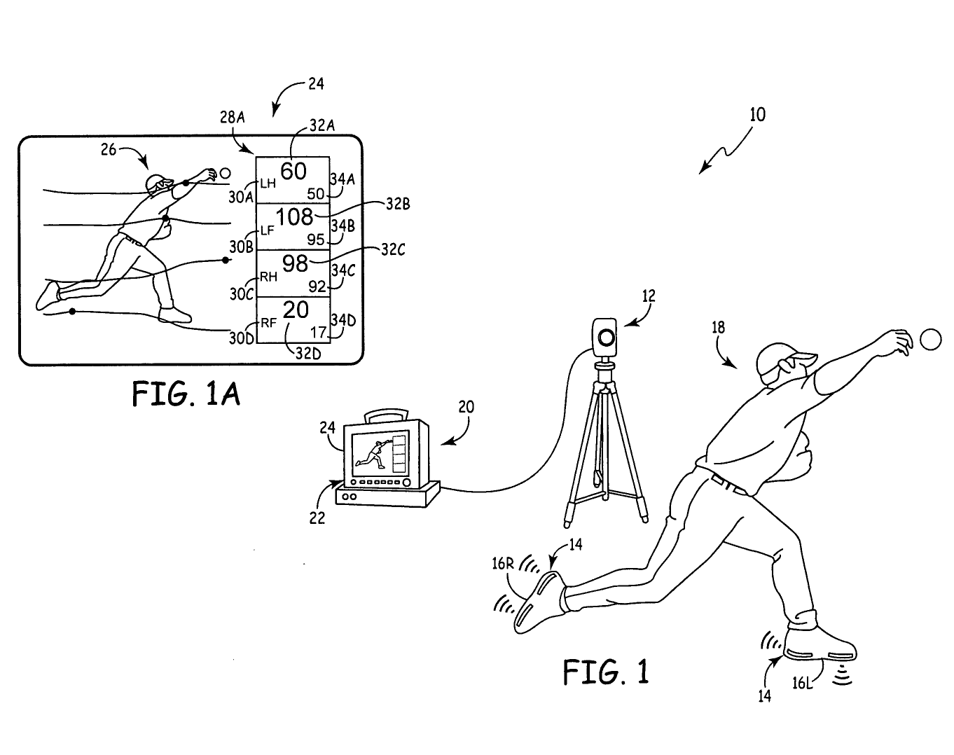

FIG. 1 shows one embodiment of an athletic training assembly 10 that synchronizes video signal captured by a video unit 12 with force data measured by a sensor assembly 14 worn in the insoles 16L and 16R of an athlete 18. In addition to the video unit 12 and the sensor assembly 14, the athletic training assembly 10 includes a main unit 20 that has a user interface 22 with a display 24.

As shown in FIG. 1, the video unit 12 records biomechanics of the athlete 18 performing various movements which simulate those performed by the athlete during athletic competition. Simultaneous with the recording of the biomechanics of the athlete 18, the sensor assembly 14 in the left and right insoles 16L and 16R measures the ground reaction forces generated by the feet of the athlete 18 striking the ground. Both the video and the measure ground reaction forces are converted to video signal and force data and are sent to the main unit 20. The main unit 20 receives both the video signal and force data and synchronizes this information. The main unit 20 also stores the video signal and force data for later recall and analysis by the operator. The synchronized video signal and force data can be selectively shown and manipulated on the display 24 by the operator using the user interface 22.

In the embodiment shown in FIG. 1, the athlete 18 is a baseball pitcher and the athletic training assembly 10 generates and collects image and force sample data during various phases of a pitching cycle (cocking, windup, delivery and follow through). The operator (for example, a coach, trainer, or sports medicine professional) can review the synchronized video signal and force data and determine lack of specific strength by the pitcher in the various phases his pitching cycle. The coach, trainer, or sports medicine professional can use the synchronized force data and video signal to correct faulty movement patterns, to spot timing and power peaks, and to ensure the athlete is maximizing his/her anatomical potential. The athletic training assembly 10 allows for storage of synchronized data over an extended time period. Stored data can be used to determine if the athlete continues to make progress with chronological change as well as physical maturity.

Although a baseball player is shown in FIG. 1, the athletic training assembly 10 is useful to study and improve the biomechanics of athletes from a variety of sports including basketball, football, golf, tennis, and track and field. For example, in football, a quarterback's ability to drop into a step pattern and apply a quick release to the ball is the hallmark of an effective player at this position. The athletic training assembly 10 will allow the coach or trainer to chart the forces that develop as the player goes through the step pattern and throwing motion. This analysis can be used to identify poor habits, such as a slow drop and throwing off the back foot.

In tennis, it is a common understanding that service velocity is directly correlated with leg strength and power. The athletic training assembly 10 can record the forces exerted during serve action and then allow the coach or trainer to relay this information to the player in a short biofeedback loop allowing the athlete to receive and absorb instruction at a much faster rate than normal verbal reinforcement. The athletic training assembly 10 allows the coach to have verbal and visual feedback at their fingertips to impart instruction to the player.

FIG. 1A shows one embodiment of the display 24 of the main unit 22 of the athletic training assembly 10. The display 24 renders an image 26. The display 24 also shows a numerical display 28 of forces measured by the sensor assembly 14. In one embodiment, the numerical display 28 shows sensor location 30A-30D, maximum force 32A-32D, and instantaneous force 34A-34D for that video frame.

Upon force/biomechanics session completion or alternatively in real time, the user interface 22 allows the operator to show initial results on the display 24. The display 24 includes the image 26 of the athlete and can additionally include one or more graphs charting the ground forces developed by the athlete as measured by the sensor assembly 14. The numerical display 28 shows the location 30A-30D of the sensors in the shoe (in the embodiment shown the sensors are located in the left heal (LH), right heal (RH), left forefoot (LF), and right forefoot (RF)). The numerical display 28 also shows the maximum force 32A-32D measured by each sensor and the instantaneous force 34A-34D measured during the corresponding nearest video frame image 26 being displayed.

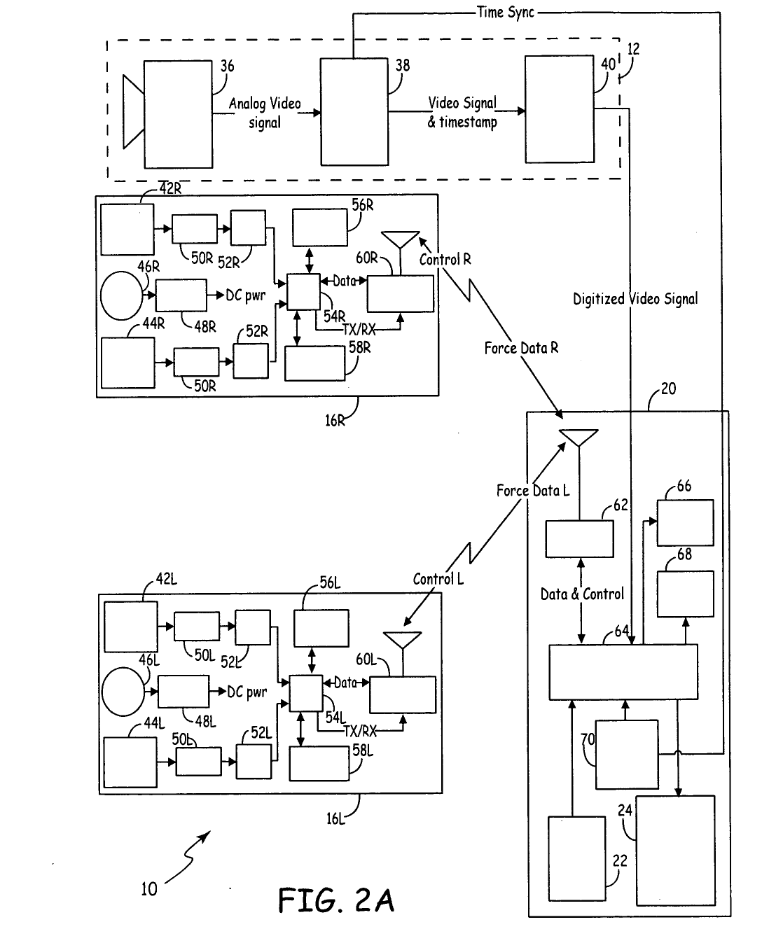

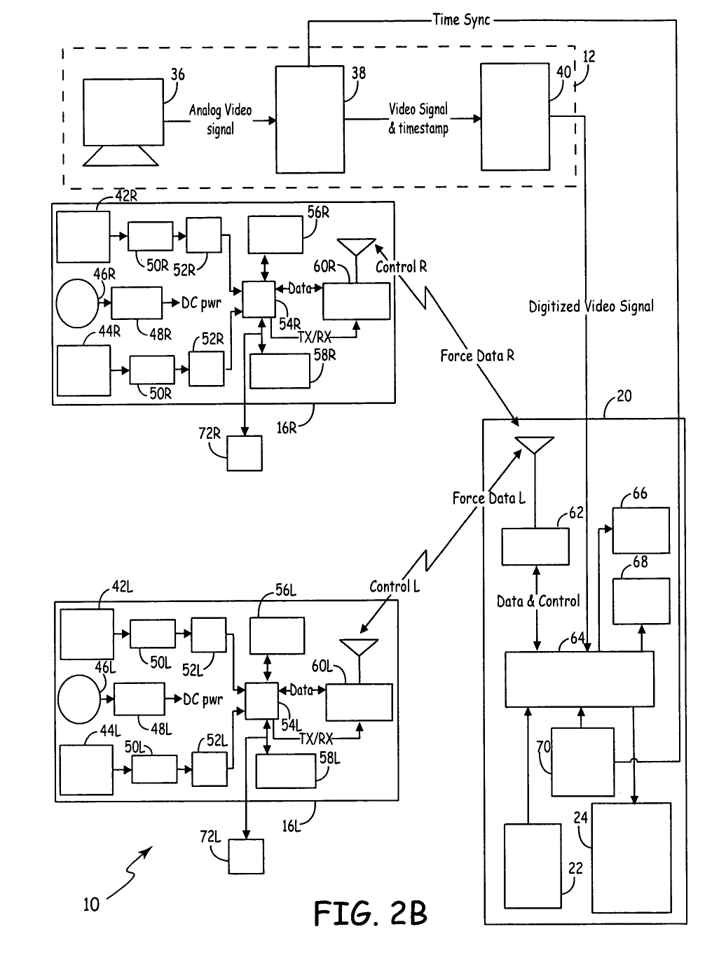

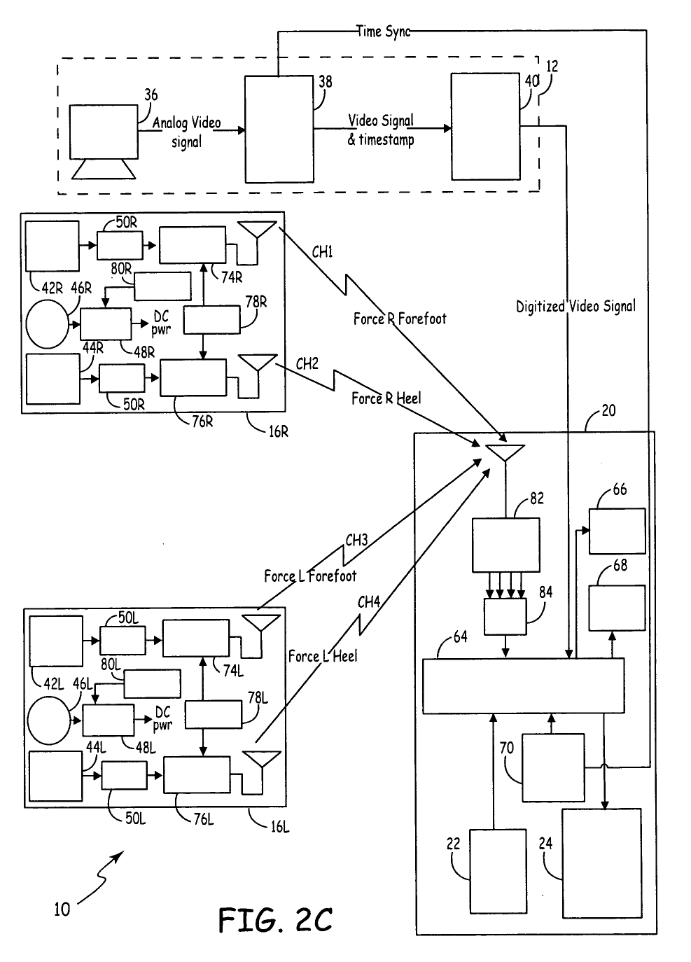

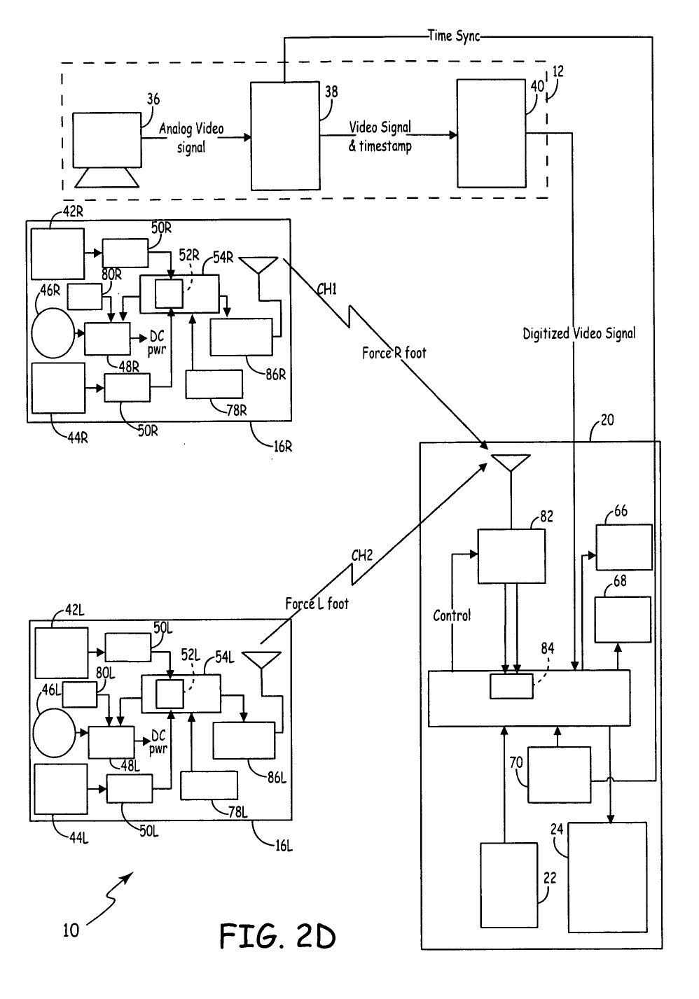

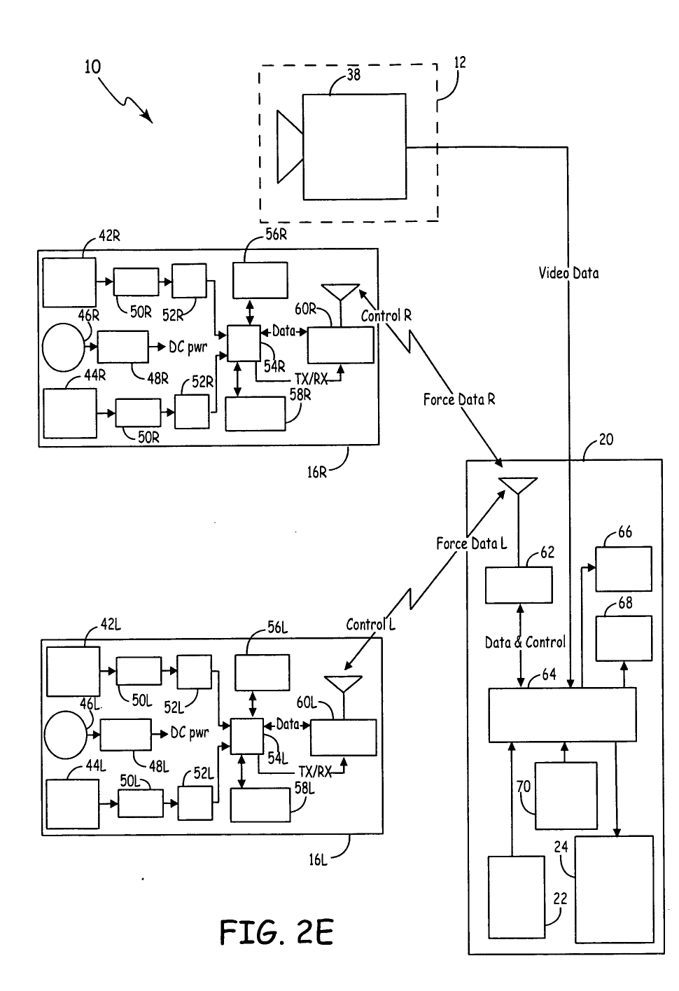

FIGS. 2A-2E are block diagrams showing various embodiments of the athletic training assembly 10 with associated components. In FIG. 2A, the video unit 12 includes a video camera 36, a video time inserter 38, and a video grabber 40. The left insole 16L and the right insole 16R house the sensor assembly 14. In one embodiment, the sensor assembly 14 includes left and right forefoot force sensors 42L and 42R and left and right-heel force sensors 44L and 44R. The left and right insoles 16L and 16R also include batteries 46L and 46R, power controllers 48L and 48R, resistance-to-voltage converters 50L and 50R, analog-to-digital converters 52L and 52R, central processing units (CPU) 54L and 54R, which include data buffering 56L and 56R regions of memory, reference timers 58L and 58R, and transceivers 60L and 60R. In addition to the user interface 22 and the display 24, the main unit 20 includes a main transceiver 62, a main CPU 64, which includes image storage 66 and force data storage 68 regions of memory, and a reference clock 70.

The video unit 12, left insole 16L, and right insole 16R are adapted to transmit data samples (force or video) to the main unit 20, and in the case of the left and right insoles 16L and 16R, to receive control signals therefrom. The main unit 20 is configured to receive the video and force data samples and synchronize the received data for presentation on the display 22 in real time to the observer. Additionally, the main unit 20 can store the received data for later recall, and is responsive to commands by the operator via the user interface 24.

One or more video cameras 36 capture analog video images of the athlete and send that video signal to the video time inserter 38. The video time inserter 38 time stamps and transfers the video signal with the time stamps to the video grabber 40. The video grabber 40 accumulates a frame of video with the associated time stamp and passes it on to the main unit 20 as a digitalized video signal.

One of the insoles 16L and 16R is installed in each shoe of the athlete 18 (FIG. 1). Within each insole 16L and 16R is a power source, and sufficient circuitry to measure a series of ground reaction force samples. Each insole 16L and 16R is also configured to report (either in a wireless manner or in a wired manner) the force measurements to the main unit 20.

In one embodiment, the senor assembly 14 is comprised of the left and right forefoot force sensors 42L and 42R, which are installed in the forward part of the insoles 16L and 16R, and the left and right heel force sensors 44L and 44R, which are installed beneath the heel of the athlete 18 (FIG. 1). In other embodiments, the sensor assembly 14 can include additional sensors in each insole 16L and 16R which would allow higher resolution force mapping. Implicit in FIGS. 2A-2E is that one signal is generated by each sensor 42L, 42R, 44L, and 44R, for a total of four force data channels between the two insoles 16L and 16R. However, a signal architecture of up to three signals per sensor 42L, 42R, 44L, and 44R for a total of up to twelve data channels can also be used. The additional signals would allow for better detection of xyz forces and/or accelerations (i.e. along the medial-lateral and anterior-posterior, as well as the normal) directions.

In the embodiment shown, the left and right forefoot force sensors 42L and 42R and left and right heel force sensors 44L and 44R are devices that operate by changing resistance as applied force is varied. Thus, these force sensors 42L, 42R, 44L, and 44R are piezoresistive. However, in other embodiments force, load, or acceleration sensors other than resistive type could be used; assuming their packaging is consistent with insole installation. For example, the sensors 42L, 42R, 44L, and 44R could be piezoelectric (i.e. vary their output voltage rather than their resistance) or accelerometers. Either of these examples would eliminate the need for the resistance-to-voltage converter 50L and 50R.

If the insoles 16L and 16R transmit and receive data wirelessly as shown in FIG. 2A, the battery 46L and 46R in each insole 16L and 16R provides operational power to components shown. The power controller 48L and 48R controls the amount of dc power supplied to the components of the insoles 16L and 16R. The power controller 48L and 48R provides for a sleep mode, so that the components reduce power consumption from each battery 46L and 46R during periods between operation (i.e. activity by the athlete).

The increased resistance experienced by the left and right forefoot force sensors 42L and 42R and left and right heel force sensors 44L and 44R as force is applied is converted to a voltage by the resistance-to-voltage converter 50L and 50R. The analog voltage signal is converted to a digital signal (representing force data) by the analog-to-digital converter 52L and 52R (which maybe part of the CPU 54L and 54R). From the analog-to-digital converter 52L and 52R the signal originating in the left and right forefoot force sensor 42L and 42R or the left and right heel force sensor 44L and 44R is passed to the CPU 54L and 54R.

The force data is buffered in CPU 54L and 54R memory, specifically in the buffering 56L and 56R region, before be passed on to transceiver 60L and 60R. In FIG. 2A, the reference timer 58L and 58R stamps the force data before it is sent to buffering 56L and 56R region.

In FIG. 2A, the transceiver 60L and 60R is configured to transmit and receive data wirelessly using radio frequency signals. As shown, the transceivers 60L and 60R utilize a single half-duplex RF channel; however, other RF architecture is also contemplated. In addition to transferring force data to the transceiver 60L and 60R, the CPU 54L and 54R also provides a control signal to the transceiver 60L and 60R to tell it to receive (RX) command signals from the main unit 20 or transfer (TX) signals to the main unit 20. Thus, command signals can be received by the transceiver 60L and 60R and then transferred to the CPU 54L and 54R. These command signals can include a command to awake from the sleep mode. Additionally, the command signals can reset the reference timer 58L and 58R so that reference timer 58L and 58R operates from the same time base as that utilized by the video time inserter 38 and main unit 20.

The main transceiver 62 receives force data from both the transceivers 60L and 60R and passes this information to the main CPU 64. As alluded to earlier, the main transceiver 62 also receives control commands from the main CPU 64 and transmits them to the transceivers 60L and 60R.

The main CPU 64 receives the force data and the video signal and has the ability to synchronize (using the data's time stamps) and store the data in image storage 66 and force data storage 68 regions its of memory. The main CPU 64 also receives control commands from the user interface 22 and sends the synchronized data to the display 24 and associated driver. Optionally, prior to storage, the main CPU 64 can resynchronize the force data utilizing the time stamps provided by the reference timer 58L and 58R to the time base used by the main unit 20 and the video time inserter 38. The time base used by the main unit 20 is provided by the reference clock 70. The reference clock 70 can be part of the main CPU 64 or can be a stand alone unit. In the embodiment shown, the reference clock 70 is hard-wired to the video time inserter 38 such that both units have the same base time. Alternatively, the same base time can be achieved by the use of off the shelf GPS time references at both the video time inserter 38 and the main unit 20. Such a design would eliminate the need for the hard-wire.

In one sequence of operation, the athletic training assembly 10 the insoles of the athlete's shoes are replaced with insoles 16L and 16R. The video camera(s) 36 is adjusted to capture the athlete's image on the main unit 20 display 24 while he or she performs the desired biomechanical movements. The operator, stationed at the main unit 20, commands the video unit 12 and sensor assembly 14 to begin a recording session via the user interface 22. The operator then instructs the athlete to perform his or her biomechanical movement. Upon the start command, the main unit 20 via main transceiver 62 resynchronizes the reference timers 58L and 58R to the time base used by that of the video time inserter 38. The main unit 20 begins collecting image frame data from the video grabber 40. At the same time, the force sensors 42L, 42R, 44L, and 44R are being sampled and the results (force data) sent to the main unit 20. Prior to storage, the main unit 20 can optionally archive the synchronized data by using the video signal's timestamps. The recording session can be ended via operator command, or alternatively, by expiration of a predetermined time-out period. The display 24 can show the initial or real time synchronized image and force data to the operator. Corrective action for the athlete may then by suggested by the operator after observation of the displayed results. Corrective action could also be suggested after comparison of current results with a known good baseline for the athlete under test or for other athletes in that sport.

FIG. 2B illustrates another embodiment of the athletic training assembly 10. In most respects the embodiment shown is similar to the embodiment shown in FIG. 2A. The FIG. 2B embodiment differs in that each shoe has an LED 72L and 72R installed on the outside thereof. The LEDs 72L and 72R are affixed to the outside of the shoes in positions within sight of the video camera 36. After all the necessary preparations discussed in FIG. 2A have been completed and prior to when the athlete is instructed to begin his or her biomechanical movements, the operator's start command is relayed via the main unit 20 to the insoles 16L and 16R. The start command can wakeup the components of the insoles 16L and 16R and initiate sampling by the force sensors 42L, 42R, 44L, and 44R. The insoles 16L and 16R via transceivers 60L and 60R respond to the main unit 20, confirming reception of the start command. The insoles 16L and 16R then simultaneously flash the LEDs 72L and 72R, insert time stamps within the force data, and reset the synchronization reference timers 58L and 58R. The main unit 20 is adapted to detect the video frames containing the LED pulses. Using the time stamps provided by the video time inserter 38 or the LED pulses, the main unit 20 determines the synchronization between the image and force data streams.

FIG. 2C shows a third embodiment of the athletic training assembly 10. The insoles 16L and 16R additionally include first transmitters 74L and 74R, second transmitters 76L and 76R, channel selection switches 78L and 78R, and pressure or load sensors 80L and 80R. The main unit 20 additionally includes a multi-channel receiver 82 and a analog-to-digital converter 84.

In FIG. 2C, the transceivers 60L and 60R have been replaced by first and second transmitters 74L, 74R, 76L, and 76R. Thus, each force sensor 42L, 42R, 44L, and 44R in that insoles 16L and 16R uses one simplex RF channel. This arrangement allows four analog signals to be transmitted to the multi-channel receiver 82, and eliminates the need for some of the components shown in FIGS. 2A and 2B. Prior to the athlete's performing biomechanical test activities, the main unit 20 verifies the integrity of the RF links and displays the results to the operator. If interference is detected, the athlete or operator can change RF channel frequencies using manual channel selection switches 78L and 78R in the insoles 16L and 16R. Channel selection switches 78L and 78R must be manually adjusted since the transmitters 74L, 74R, 76L, and 76R cannot receive control commands from the main unit 20. Actuation by the athlete of the pressure or load sensors 80L and 80R turns the power controllers 48L and 48R on to an operational mode from the power save sleep mode. The multi-channel receiver 82 receives the multiple RF signals and transfers them to the analog-to-digital converter 84. The analog-to-digital converter 84 converts the analog force data to digital force data for processing by the main CPU 64. The main CPU 64 applies synchronization to the video frame and force data streams, using the timestamps provided by the video time inserter 38. To simplify the synchronization process, the latency variation of the force data via the RF channels will be insignificant relative to the video frame rate.

FIG. 2D shows a fourth embodiment of the athletic training assembly 10. The insoles 16L and 16R additionally include RF transmitters 86L and 86R. The embodiment shown in FIG. 2C combines several components of the embodiments shown in FIGS. 2A and 2B with the channel selection switches 78L and 78R of FIG. 2C. The signals generated by the force sensors 42L, 42R, 44L, and 44R within insoles 16L and 16R are digitized and then time division multiplexed into a serial steam of force data. The force data stream within each insole 16L and 16R then provides the modulation signal for the RF transmitters 86L and 86R. The transmitters 86L and 86R transmit RF signals on two channels which are received by the multi-channel receiver 82. The multi-channel receiver 82 receives the multiple RF signals and transfers them to the analog-to-digital converter 84. Channel selection switches 78L and 78R must be manually adjusted since the transmitters 74L, 74R, 76L, and 76R cannot receive control commands from the main unit 20. Actuation by the athlete of the pressure or load sensors 80L and 80R turns the power controllers 48L and 48R on to an operational mode from the power save sleep mode. The main unit 20 synchronizes the video frame and force data streams, using the timestamps provided by the video time inserter 38.

FIG. 2E shows a fifth embodiment of the athletic training assembly 10. In most respects the embodiment shown is similar to the embodiment shown in FIG. 2A. The FIG. 2E embodiment differs in that the video time inserter 38 and the video grabber 40 have been removed from video unit 12. Thus, prior to storage in the memory of the main CPU 64, the main unit 20 synchronizes the video signal and the force data by compensating for known delays in the video and force sample paths.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. An assembly for optimizing performance of an athlete, comprising:
a video unit configured to capture an image of the athlete and generate video signal thereof;
a sensor assembly worn in a shoe of the athlete to measure ground reaction forces generated by the athlete;
a transmission device that transmits force data representative of the measured ground reaction forces; and
a main unit adapted to receive and synchronize both the force data and the video signal.

2. The apparatus of claim 1, wherein the sensor assembly includes a first sensor installed in a right insole of a right shoe of the athlete and a second sensor installed in a left insole of a left shoe of the athlete.

3. The apparatus of claim 2, wherein the first sensor has a forefoot force sensor and a heel force sensor and the second sensor has a forefoot force sensor and a heel force sensor.

4. The apparatus of claim 2, wherein the first sensor has multiple force sensors and the second sensor has multiple force sensors.

5. The apparatus of claim 1, wherein the video unit comprises:
a video camera that generates the video signal;
a video time inserter which time stamps the video signal; and
a video grabber that accumulates a frame of video signal with associated time stamp and passes the frame of video signal and associated time stamp along to the main unit.

6. The apparatus of claim 1, further comprising an insole reference timer which is installed in the shoe of the athlete, the insole reference timer is responsive to control signals to time stamp the force data.

7. The apparatus of claim 5, wherein the main unit synchronizes force data with video signal using the time stamps of the video signal.

8. The apparatus of claim 1, wherein the main unit synchronizes force data with video signal by compensating for known delays in image and force sample paths.

9. The apparatus of claim 7, further comprising an LED which is installed in the shoe of the athlete, the LED is responsive to control signals to flash thereby allowing the main unit to synchronize force data with video signal.

10. The apparatus of claim 1, wherein the transmission device is an RF transceiver which receives control commands from the main unit.

11. The apparatus of claim 1, wherein the synchronized video signal and force data can be selectively shown on a display of the main unit.

12. The apparatus of claim 1, wherein the main unit collects and stores video signal and force data for later recall and display.

13. The apparatus of claim 11, wherein the display of the main unit shows video images and force data to the observer in real time.

14. A method of optimizing performance of an athlete, comprising:
capturing an image of the athlete performing a biomechanical movement;
generating a video signal from the captured image;
measuring ground reaction forces generated by the athlete in insoles during the biomechanical movement;
transmitting force data representative of the measured ground reaction forces to a central processing unit; and
synchronizing the force data with the video signal by the central processing unit for display or storage.

15. The method of claim 14, wherein the measured ground forces are time synchronized to the video signal.

16. The method of claim 14, wherein the central processing unit synchronizes force data with video signal by compensating for known delays in image and force sample paths.

17. A method of optimizing performance of an athlete, comprising:
measuring ground forces generated by the athlete; and
correlating the measured ground forces with movements of the athlete.

18. The method of claim 17, wherein the movements of the athlete are captured by a video camera which generates a video signal.

19. The method of claim 18, wherein the measured ground forces are time synchronized to the video signal.

20. The method of claim 17, wherein the step of correlating the measured ground forces with the movements of the athlete involves synchronizing the measured ground forces with movements of the athlete for display or storage.
